(19) United States
(12) Patent Application Publication
(10) Pub. No.: US 2022/0155325 A1
(43) Pub. Date: May 19, 2022

(54) METHOD FOR DIAGNOSING ALZHEIMER'S DISEASE USING SILVER NANOGAP SHELL

(71) Applicant: INDUSTRY-UNIVERSITY COOPERATION FOUNDATION HANYANG UNIVERSITY ERICA CAMPUS, Ansan-si (KR)

(72) Inventors: Jong Ho KIM, Ansan-si (KR); Jin-Kyoung YANG, Ansan-si (KR); In Jun HWANG, Ansan-si (KR); Hye In KIM, Suwon-si (KR)

(73) Assignee: INDUSTRY-UNIVERSITY COOPERATION FOUNDATION HANYANG UNIVERSITY ERICA CAMPUS, Ansan-si (KR)

(21) Appl. No.: 17/436,710

(22) PCT Filed: Mar. 6, 2020
(86) PCT No.: PCT/KR2020/003145

§ 371 Date: Sep. 7, 2021

(30) Foreign Application Priority Data
Mar. 6, 2019 (KR) 10-2019-0025702
Mar. 5, 2020 (KR) 10-2020-0027910

(51) Int. Cl. G01N 33/68; G01N 33/543; G01N 33/553; G01N 21/65

(52) U.S. Cl. CPC G01N 33/6896; G01N 33/54346; G01N 2800/2821; G01N 21/658; G01N 2333/4709; G01N 33/553

(57) ABSTRACT

The present disclosure relates to a method for diagnosing Alzheimer's disease (AD) using a silver nanogap shell, which enables non-invasive early diagnosis of AD via multiplexed detection of a plurality of Alzheimer's disease target biomarkers with high sensitivity.

### TECHNICAL FIELD

The present disclosure relates to a method for diagnosing Alzheimer's disease using a silver (Ag) nanogap shell (AgNGS). More particularly, the present disclosure relates to a method for diagnosing Alzheimer's disease using silver nanogap shell-based SERS immunoassay, which enables non-invasive early diagnosis of Alzheimer's disease via multiplexed detection of a plurality of Alzheimer's disease target biomarkers with high sensitivity.

### BACKGROUND ART

The diagnosis of Alzheimer's disease (AD) in clinical practice has relied on brain imaging techniques such as PET, etc., medical interviews, and enzyme-linked immunosorbent assay (ELISA) of Aβ40, Aβ42, etc. present in the cerebrospinal fluid. Recently, various diagnostic methods based on nanotechnology have been developed for accurate and early diagnosis of AD. In particular, although the SERS-based diagnostic method receives a lot of attentions in terms of sensitivity and multiplexed detection, the SERS-based diagnostic method developed thus far has the problem that is limited to detection of a single target and the sensitivity is very low. Therefore, researches are ongoing to develop a SERS-based method for diagnosing AD, which is capable of detecting a plurality of target biomarkers in blood with high sensitivity.

The existing methods require expensive equipment and involve patient's suffering due to invasive cerebrospinal fluid sampling. In addition, they have the fatal problem that diagnosis is possible only after symptoms have progressed considerably because the progression of disease cannot be traced. Furthermore, they are limited in that they trace only a single biomarker although it is necessary to trace a plurality of related biomarkers at the same time for reliable, accurate and early diagnosis of AD. Besides, a technology of detecting a plurality of biomarkers from a sample of a patient such as blood, etc. via a noninvasive or minimally invasive method is required for early diagnosis. For this, a very sensitive diagnostic technology is necessary since AD target biomarkers are present in a blood sample in trace amounts of pg/mL scale. However, the required sensitivity cannot be achieved with the existing technology such as ELISA. Accordingly, a technology for noninvasive multiplexed detection and early diagnosis of AD with high sensitivity is still needed.

### DISCLOSURE

### Technical Problems

The present disclosure is directed to providing a composition for diagnosing Alzheimer's disease, which contains a silver nanogap shell capable of detecting a plurality of biomarkers for diagnosis of Alzheimer's disease at the same time.

The present disclosure is also directed to providing a method for diagnosing Alzheimer's disease and a method for differentially diagnosing Alzheimer's disease and mild cognitive impairment using the composition.

However, the problems to be solved by present disclosure are not limited to those described above. Other problems not mentioned above will be clearly understood by those having ordinary skill in the art from the following description.

### Technical Solution

The present disclosure provides a composition for diagnosing Alzheimer's disease, which contains a silver nanogap shell with one or more antibody selected from a group consisting of an antibody specific for amyloid beta (Aβ) 40 and antibody specific for Aβ42 introduced as an active ingredient.

The present disclosure also provides a biosensor for diagnosing Alzheimer's disease, which includes a silver nanogap shell with an antibody specific for Aβ40 or Aβ42 introduced and a substrate on which the silver nanogap shell is immobilized.

In an exemplary embodiment of the present disclosure, the substrate may include a magnetic bead and the silver nanogap shell may be immobilized using the magnetic bead.

The present disclosure also provides a method for diagnosing Alzheimer's disease and a method for providing information for diagnosis of Alzheimer's disease, which include:

(1) a step of preparing AgNGS-Aβ40 by conjugating an antibody specific for amyloid beta (Aβ) 40 on the surface of a silver nanogap shell (AgNGS); (2) a step of preparing AgNGS-Aβ42 by conjugating an antibody specific for Aβ42 on the surface of AgNGS; (3) a step of inducing the formation of a silver nanogap shell (AgNGS)-based sandwich complex by mixing the AgNGS-Aβ40 and the AgNGS-Aβ42 with a biological sample in vitro; (4) a step of calculating a ratio of Aβ40 and Aβ42 by measuring a Raman signal of the complex in the sample; and (5) a step of diagnosing as Alzheimer's disease when the ratio of Aβ40 and Aβ42 is from 2.5 to 999.

In an exemplary embodiment of the present disclosure, the biological sample may be blood or serum, specifically serum.

The present disclosure also provides a composition for differentially diagnosing Alzheimer's disease and mild cognitive impairment, which contains a silver nanogap shell with an antibody specific for Aβ40 introduced (AgNGS-Aβ40) and a silver nanogap shell with an antibody specific for Aβ42 introduced (AgNGS-Aβ42) as active ingredients.

The present disclosure also provides a biosensor for differentially diagnosing Alzheimer's disease and mild cognitive impairment, wherein the AgNGS-Aβ40 and the AgNGS-Aβ42 are immobilized on a substrate.

The present disclosure also provides a method for differentially diagnosing Alzheimer's disease and mild cognitive impairment and a method for providing information for differential diagnosis, which include:

(1) a step of preparing AgNGS-Aβ40 by conjugating an antibody specific for amyloid beta (Aβ) 40 on the surface of a silver nanogap shell (AgNGS); (2) a step of preparing AgNGS-Aβ42 by conjugating an antibody specific for Aβ42 on the surface of AgNGS; (3) a step of inducing the formation of a silver nanogap shell (AgNGS)-based sandwich complex by mixing the AgNGS-Aβ40 and the AgNGS-Aβ42 with a biological sample in vitro; (4) a step of calculating a ratio of Aβ40 and Aβ42 by measuring a Raman signal of the complex in the sample; and (5) a step of diagnosing as normal when the ratio is from −999 to 1.1, as mild cognitive impairment when the ratio is from 1.1 to 2.5, and as Alzheimer's disease when the ratio is from 2.5 to 999.

The present disclosure also provides a use of AgNGS-Aβ40 and AgNGS-Aβ42 for differential diagnosis of Alzheimer's disease and mild cognitive impairment.

### Advantageous Effects

According to a method for diagnosing Alzheimer's disease using a silver nanogap shell according to the present disclosure, two amyloid peptides (Aβ40 and Aβ42) can be detected at the same time by immunoassay using an AgNGS nanoprobe which exhibits a characteristic surface-enhanced Raman scattering (SERS) signal of a marker very strongly and a magnetic bead.

In particular, the AgNGS-based SERS immunoassay enables detection of Aβ40 and Aβ42 with very high sensitivity of below 1 pg/mL, and enables simultaneous detection of a plurality of target biomarkers without interference because it reacts specifically with the respective targets.

In addition, the present disclosure enables blood-based noninvasive early diagnosis of AD through multiplexed detection of Aβ40 and Aβ42 with high sensitivity even under the complex environment of human serum. The present disclosure is also applicable to diagnosis and tracing of a plurality of diseases in addition to AD, which require high sensitivity and multiplexed detection.

### BRIEF DESCRIPTION OF DRAWINGS

FIG. 1 schematically shows immunoassay and Raman signal measurement using a magnetic bead modified with a target-specific antibody and AgNGS.

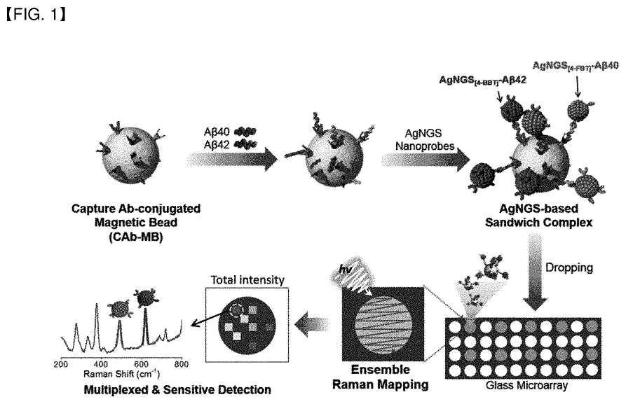

FIG. 2 shows a detection result for two Alzheimer's disease (AD) biomarkers (Aβ40, Aβ42) (a): microarray mapping images for Aβ40 detection depending on concentrations, b): Raman signals of AgNGS at regions marked in a), (c): signals for different concentrations in a), d): microarray mapping images for Aβ42 detection depending on concentrations, e): Raman signals of AgNGS at regions marked in d), f): signals for different concentrations in d)).

FIG. 3 shows the verification of the specificity of AgNGS-based SERS immune response.

FIG. 4 shows the ability of multiplexed detection in a blood sample.

FIG. 5 shows the detection result of Aβ40 and Aβ42 in a clinical sample.

### BEST MODE

The inventors of the present disclosure have researched on a technology for diagnosing AD early by detecting a plurality of biomarkers from a noninvasive sample with high sensitivity, and have completed the present disclosure.

Alzheimer's disease (AD) is a neurodegenerative brain disease. The most common early symptom is difficulty in remembering recent events. As the disease advances, symptoms can include decline in various cognitive functions. Amyloid plaques and neurofibrillary tangles are observed in the brain tissue of a patient with Alzheimer's disease, and brain atrophy due to the loss of neurons is observed in the brain MRI image. Although they are confined in the early stage in the hippocampus and the entorhinal cortex, which are important bran parts responsible for memory, they gradually spread through the brain, including the parietal lobe, the frontal lobe, etc. Especially, it is known the apolipoprotein E (APOE) epsilon 4 allele gene increases the prevalence rate of Alzheimer's disease. It is known that other APOE epsilon genes except epsilon 4 inhibit the production of amyloid beta proteins (Aβ proteins). The inventors of the present disclosure have identified that biomarkers for diagnosis of Alzheimer's disease can be detected with very high sensitivity from a noninvasive sample (particularly, serum) by using silver (Ag) nanogap shell (AgNGS)-based SERS immunoassay and two or more biomarkers can be detected at the same time.

Therefore, the present disclosure may provide a composition for diagnosing Alzheimer's disease, which contains a silver nanogap shell with an antibody specific for Aβ40 and/or Aβ42 introduced as an active ingredient.

The composition of the present disclosure may further contain, in addition to the antibody-introduced silver nanogap shell, a pharmacologically or physiologically acceptable carrier, excipient or diluent. Examples of suitable carrier, excipient or diluent that may be contained in the composition include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinylpyrrolidone, water, methyl hydroxybenzoate, propyl hydroxybenzoate, talc, magnesium stearate, mineral oil, etc. When the composition is prepared into a drug, it may further contain a commonly used filler, extender, binder, disintegrant, surfactant, anticoagulant, lubricant, wetting agent, flavorant, emulsifier, antiseptic, etc.

In the present disclosure, the silver (Ag) nanogap shell (AgNGS) is a nanoprobe consisting of: a silica particle as a base particle; and a silver (Ag)-containing metal layer surrounding the silica particle and having a plurality of gaps, wherein a Raman label is introduced in the silver gaps of the metal layer to emit surface-enhanced Raman scattering signals and an antibody specific for Aβ40 or Aβ42 is introduced on the surface of the metal layer, thereby forming AgNGS-Aβ40 or AgNGS-Aβ42. The antibody included in AgNGS-Aβ40 or AgNGS-Aβ42 forms a silver nanogap shell (AgNGS)-based sandwich complex by binding to Aβ40 or Aβ42 contained in the sample with high specificity. The inventors of the present disclosure have identified that Aβ40 and/or Aβ42 can be detected with very high sensitivity by detecting the surface-enhanced Raman scattering (SERS) signal of the complex.

Meanwhile, mild cognitive impairment (MCI) which refers to a condition where cognitive function is below the normal level is known as a prodromal stage of Alzheimer's disease. It refers to a condition where the activities of daily living are not interfered with. Patients with mild cognitive impairment are highly likely to progress to Alzheimer's disease. However, it is known that 25-30% of patients with mild cognitive impairment recover if appropriate treatment is given in the early stage. For prevention of Alzheimer's disease and fast treatment after onset, early diagnosis of mild cognitive impairment is necessary. And, for adequate treatment, differential diagnosis of mild cognitive impairment and Alzheimer's disease is also necessary.

The inventors of the present disclosure have induced complex formation by reacting the serum samples of normal people or patients diagnosed with mild cognitive impairment or Alzheimer's disease with AgNGS-Aβ40 or AgNGS-Aβ42 of the present disclosure and calculated the ratio of Aβ40 and Aβ42 from SERS signals according to Equation 1.

$$A β40 \text{/} A β42 = {\sum }_{1}^{n > 0} {I}_{320 - 430} \text{/} {\sum }_{1}^{n} {I}_{450 - 510} \qquad [ Equation 1 ]$$

I_{A-B}=the sum of Raman intensities in range from A to B

As a result, it was found out that the samples from the normal people, patients with mild cognitive impairment and patients with Alzheimer's disease have different Aβ40/Aβ42 values (smaller values for cognitive ability; larger values for cognitive problem). Specifically, the Aβ40/Aβ42 value was in a range from 1.1 to 2.5 for mild cognitive impairment. Smaller values could be diagnosed as normal and higher values could be diagnosed as Alzheimer's disease.

Therefore, the present disclosure enables fast and accurate differential diagnosis of dementia and mild cognitive impairment and enables timely and adequate treatment of dementia early diagnosis by providing an accurate diagnosis result using a noninvasive sample (serum).

### MODE FOR INVENTION

The present disclosure may be changed variously and may have various exemplary embodiments. Hereinafter, the specific exemplary embodiments will be described in detail. However, the present disclosure is not limited to the specific exemplary embodiments and it should be understood to include all changes, equivalents and substitutes within the technical idea and scope of the present disclosure. When describing the present disclosure, detailed description of known technology will be omitted if it obscures the gist of the present disclosure unnecessarily.

### EXAMPLES

### Example 1. Preparation of AgNGS-Aβ40 and AgNGS-Aβ42

Silver nanogap shells with an antibody specific for Aβ40 or Aβ42 introduced independently were prepared as follows (see KR 10-1944346).

1-1. Synthesis of Silica Particle and Formation of Thiol Functional Group

(1) After dissolving tetraethyl orthosilicate in 40 mL of absolute ethanol, ammonium hydroxide was added and reaction was conducted at room temperature for 20 hours.

(2) After removing unreacted materials by centrifugation, the reaction mixture was diluted to a final concentration of 1 mg/mL.

(3) After adding mercaptopropyltrimerhoxysilane and ammonium hydroxide to 1 mL of the synthesized silica particle (1 mg/mL), reaction was conducted at room temperature for 12 hours.

(4) Unreacted materials were removed by centrifugation (MPTS-silica).

1-2. Formation of Silver Nanogap Shell on Silica Particle Surface

(1) After dissolving MPTS-silica, PVP and AgNO₃ in 50 mL of ethylene glycol, reduction was conducted by adding octylamine. A Raman label (4-fluorethiophenol, 4-bromothiophenol, 4-chlorothiophenol or benzenethiol) was added 1 minute after the addition of octylamine.

(2) Unreacted materials were removed by centrifugation (AgNGS).

1-3. Formation of Self-Assembled Monolayer Using Mercaptoundecanoic Acid and Mercaptohexanol for Introduction of Functional Group onto Surface of Silver Nanogap Shell

(1) After adding mercaptohexanol and mercaptoundecanoic acid to the synthesized AgNGS, reaction was conducted in ethanol for 1 hour.

(2) Unreacted materials were removed by centrifugation (SAM-AgNGS).

1-4. Introduction of Antibody Binding Selectively to Aβ40 or Aβ42 onto Surface of Silver Nanogap Shell

(1) After adding an EDC/NHS (1-ethyl-3-(3-dimethylaminopropyl)carbodiimide/N-hydroxysuccinimide) coupling agent to the synthesized SAM-AgNGS, reaction was conducted in a 100 mM PBS solution of pH 6.0 for 30 minutes.

(2) Unreacted materials were removed by centrifugation.

(3) After adding an antibody binding selectively to Aβ40 or Aβ42 (1 mg/mL, 10 L), reaction was conducted in a 100 mM PBS buffer of pH 7.4 for 2 hours.

(4) After removing unreacted materials by centrifugation, the reaction mixture was dispersed in a 1% BSA solution and then stored in a refrigerator.

### Example 2. Detection of Two Alzheimer's Disease (AD) Biomarkers (Aβ40, Aβ42)

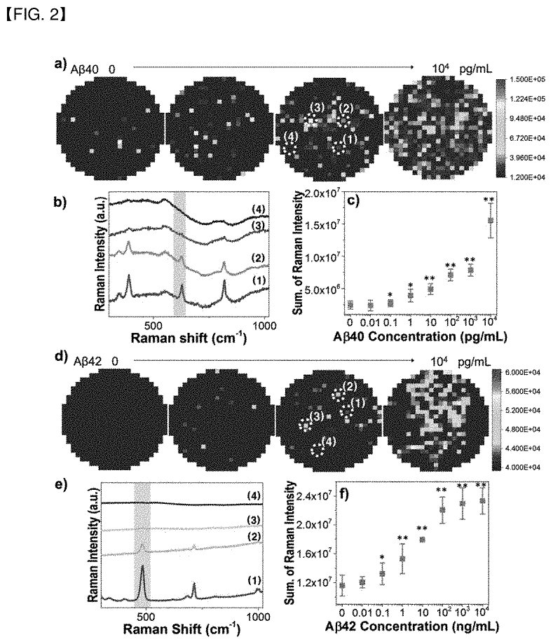

FIG. 2 shows a detection result for two Alzheimer's disease (AD) biomarkers (Aβ40, Aβ42) (a): microarray mapping images for Aβ40 detection depending on concentrations, b): Raman signals of AgNGS at regions marked in a), c): signals for different concentrations in a), d): microarray mapping images for Aβ42 detection depending on concentrations, e): Raman signals of AgNGS at regions marked in d), f): signals for different concentrations in d)).

Referring to FIG. 2, it can be seen that Aβ40, Aβ42 can be detected by AgNGS-based SERS immunoassay in a concentration-dependent manner.

### Example 3. Verification of Specificity of AgNGS-Based SERS Immune Response

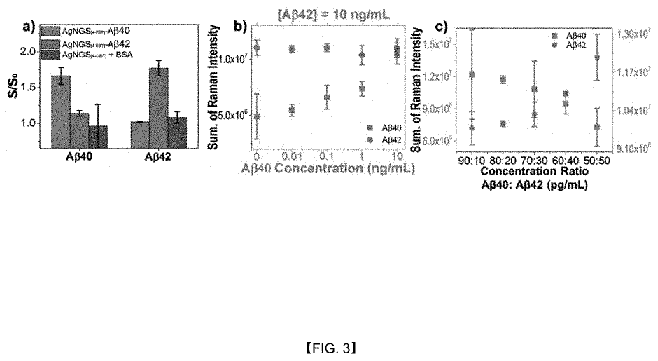

FIG. 3 shows the verification of the specificity of AgNGS-based SERS immune response.

In FIG. 3, a) shows the Raman signals from AgNGS modified with the antibody specific for each target biomarker. b) shows the tendency of Raman signals reflecting the change in the concentration of the two biomarkers in AgNGS-based SERS immunoassay in a mixture solution of the two biomarkers with the concentration of Aβ42 fixed to 10 ng/mL and the concentration of Aβ40 changing from 0 to 10 ng/mL. c) shows the tendency of Raman signals reflecting the change in the concentration of the two biomarkers in AgNGS-based SERS immunoassay when the ratio of Aβ40 and Aβ42 is the same as that shown on the x-axis. It can be seen that the AgNGS-based SERS immunoassay is specific for a plurality of biomarkers without cross-reactivity.

### Example 4. Verification of Multiplexed Detection in Blood Sample

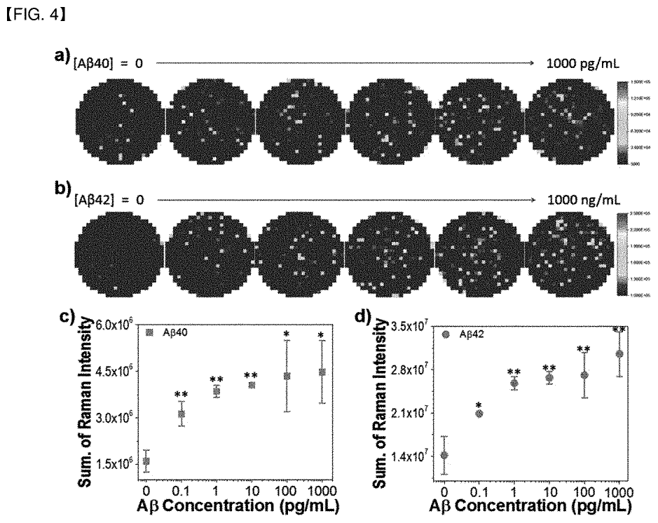

FIG. 4 shows the ability of multiplexed detection in a blood sample. Referring to FIG. 4, it can be seen that Raman signals show the tendency reflecting the change in the concentration of the two biomarkers when Aβ40 and Aβ42 are detected through AgNGS-based SERS immunoassay in a human serum sample wherein Aβ40 and Aβ42 are present at concentrations of 0-1000 ng/mL. Through this, it can be seen that Aβ40 and Aβ42 can be detected specifically from a blood sample.

### Example 5. Diagnosis Using Ratio of Aβ40 and Aβ42 in Clinical Sample

Serum samples of normal people and patients diagnosed with mild cognitive impairment or Alzheimer's disease were acquired from Boramae Hospital.

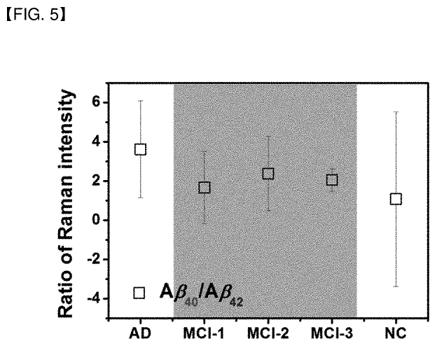

FIG. 5 shows the detection result of Aβ40 and Aβ42 in a clinical sample. Referring to FIG. 5, when the ratio of the concentrations of the two biomarkers was calculated by detecting Aβ40 and Aβ42 existing in the serum by AgNGS-based SERS immunoassay, the ratio was in a range from 1.1 to 2.5 for patients with mild cognitive impairment, below 1.1 for normal people, and higher than 2.5 for Alzheimer's disease. From this result, it can be seen that the patients with Alzheimer's disease, the patients with mild cognitive impairment (MCI) and normal people (NC) can be diagnosed differentially.

While the specific exemplary embodiments of the present disclosure have been described in detail, it will be obvious to those having ordinary knowledge in the art that they are merely specific exemplary embodiments and the scope of the present disclosure is not limited by them. Accordingly, it is to be understood that the substantial scope of the present disclosure is defined by the appended claims and their equivalents.

1. A composition for diagnosing Alzheimer's disease, comprising a silver nanogap shell with one or more antibody selected from a group consisting of an antibody specific for amyloid beta (Aβ) 40 and antibody specific for Aβ42 introduced as an active ingredient.

2. The composition for diagnosing Alzheimer's disease according to claim 1, wherein the silver nanogap shell comprises: a silica particle; and a metal layer completely surrounding the silica particle and having a surface with a plurality of gaps formed, wherein the metal layer comprises silver, a Raman label is introduced in the silver gaps and the antibodies are introduced on the surface of the metal layer.

3. (canceled)

4. The composition for diagnosing Alzheimer's disease according to claim 1, wherein the one or more antibody forms a silver nanogap shell (AgNGS)-based sandwich complex by mixed with one or more noninvasive biological sample selected from a group consisting of blood and serum.

5. A method for diagnosing Alzheimer's disease, comprising:
(1) a step of preparing AgNGS-Aβ40 by conjugating an antibody specific for amyloid beta (Aβ) 40 on the surface of a silver nanogap shell (AgNGS);
(2) a step of preparing AgNGS-Aβ42 by conjugating an antibody specific for Aβ42 on the surface of AgNGS;
(3) a step of inducing the formation of a silver nanogap shell (AgNGS)-based sandwich complex by mixing the AgNGS-Aβ40 and the AgNGS-Aβ42 with a biological sample in vitro;
(4) a step of calculating the ratio of Aβ40 and Aβ42 by measuring a Raman signal of the complex in the sample; and
(5) a step of diagnosing as Alzheimer's disease when the ratio of Aβ40 and Aβ42 is from 2.5 to 999.

6. A composition for differentially diagnosing Alzheimer's disease and mild cognitive impairment, comprising a silver nanogap shell with an antibody specific for amyloid beta (Aβ) 40 introduced and a silver nanogap shell with an antibody specific for Aβ42 introduced as active ingredients.

7. (canceled)

8. The method for diagnosing Alzheimer's disease according to claim 5, wherein the biological sample is one or more noninvasive sample selected from a group consisting of blood and serum.

9. (canceled)
